(19) United States
(12) Patent Application Publication
(10) Pub. No.: US 2013/0136669 A1
(43) Pub. Date: May 30, 2013

(54) APPLIANCE FOR DISINFECTING THE HANDS OF A USER

(71) Applicants: Bernard Feldain, Mandelieu-la-Napoule (FR); Claude Ferrand, Nice (FR)

(72) Inventors: Bernard Feldain, Mandelieu-la-Napoule (FR); Claude Ferrand, Nice (FR)

(21) Appl. No.: 13/521,975

(22) PCT Filed: Jan. 12, 2011
(86) PCT No.: PCT/FR2011/050051

§ 371 Date: Dec. 4, 2012

(30) Foreign Application Priority Data
Jan. 14, 2010 (FR) 1050225

(51) Int. Cl. A61L 2/00

(52) U.S. Cl. CPC A61L 2/0094
USPC 422/300

(57) ABSTRACT

The invention relates to an appliance for disinfecting the hands of a user, said appliance including: a disinfection chamber (1), at least one opening (10) through which the user inserts his or her hands (M) in the disinfection chamber; separators (4) placed in the disinfection chamber (1) such that the fingers are separated when the user inserts his or her hands in said chamber, characterised in that the disinfection chamber (1) is provided with a device for spraying a disinfectant product in said chamber and a device for blowing air into said chamber, said two devices operating at the same time, and in that the spraying device consists of injectors (11a, 11b, 12a, 12b) suitable for scattering a disinfectant product into the blown air stream, the scattering of the disinfectant product being carried out in a spatial sector defined by a vertical plane and an inclined plane of 1° to 90°, preferably 45° to 90°, relative to the axis of said air stream.

### FIELD OF THE INVENTION

An object of the invention is an apparatus enabling disinfection of the hands of a user.

It relates to the general technical field of sanitary apparatus and more particularly to those enabling the disinfection and the drying of hands.

### PRIOR ART

Apparatus for hand disinfection are well known in the prior art. The patent documents U.S. Pat. No. 4,942,631 (ROSA), U.S. Pat. No. 2,814,081 (STEVENSON), WO 98/53752 (DEIBERT), WO 00/48640 (SCHNEIDER), WO 2006/058374 (CHOATE) describe apparatus having a disinfection chamber with an opening enabling the user to insert his hands into the aforementioned chamber. The apparatus described in WO 98/53752 (DEIBERT) and WO 2006/058374 (CHOATE) can also comprise a dryer. Thus, a user can insert his hand(s), clean them with the cleaning and/or disinfection solution, and dry them, without any manual operation being necessary.

In general, nozzles are positioned in the disinfection chamber so as to spray a disinfectant substance on the surface of the hands. These disinfection apparatus are not, however, totally effective because they do not disinfect all parts of hands, only the front and back being treated.

Known from the document U.S. Pat. No. 3,220,424 (NELSON) is an apparatus for disinfecting the hands of a user, comprising a disinfection chamber, at least one opening through which the user inserts his hands into the disinfection chamber. Separators are in the aforementioned disinfection chamber so that the fingers separate from each other when the user inserts his hands. However, a guide for the hands is interposed between the separators and the spaying device, so that the disinfectant is not directly sprayed on the user's hands, the aforementioned guide forming a barrier reducing the effectiveness of the apparatus.

Given this state of affairs, the main objective of the invention is to provide an apparatus for disinfection more effective than those known from the prior art.

### DISCLOSURE OF THE INVENTION

The solution provided by the invention is an apparatus for the disinfection of the hands of a user, the aforementioned apparatus comprising:
a disinfection chamber,
at least one opening through which the user inserts his hands into the disinfection chamber,

This apparatus is characterized in that separators are placed in the disinfection chamber so that the fingers separate from each other when the user inserts his hands into the aforementioned chamber.

In the apparatus known from the prior art, if the user inserts his hands with the fingers closed, the disinfectant product cannot reach all contaminated areas. To the contrary, the invention enables systematic disinfection of all the places where a maximum of bacteria can accumulate, that is to say between the fingers.

To simplify the design of the apparatus, the separators are preferably vertical rods positioned in the disinfection chamber, the spacing between the aforementioned rods defining the separation of the fingers.

The decontamination chamber is advantageously equipped with two elliptical openings through which the user inserts his hands into the disinfection chamber. This elliptical shape enables minimization of the dispersions of the disinfectant product out of the chamber.

The disinfection chamber is preferably provided with a device for spraying a disinfectant product in the aforementioned chamber and a device for blowing air in the aforementioned chamber, the aforementioned two devices operating at the same time. The spraying combined with the air flow thus enables fogging, or clouding, of the disinfectant in the interior of the chamber.

The spraying device preferably comprises nozzles dispersing a disinfectant product into a stream of blown air, the aforementioned nozzles being oriented in the disinfection chamber so as to disperse the aforementioned product in a spatial sector defined by a vertical plane and an inclined plane of 1° to 90°, preferably of 45° to 90°, relative to the axis of the aforementioned air flow. Under these conditions, the disinfectant mixes product optimally mixes with air to form a homogeneous micro-fog able to more easily reach the whole surface of hands, notably between the fingers.

Advantageously, the spraying device preferably comprises nozzles dispersing a disinfectant product, the aforementioned nozzles being positioned in the disinfection chamber so as to disperse the aforementioned product onto the front and the back of hands, the dispersion being made from the end of fingers towards the wrist and from wrist towards the fingertips. It is thus possible to effectively treat the entire surface of hands.

For efficiency advantage, the separators may also be directly integrated into a device enabling spraying of disinfectant product directly between the fingers.

The disinfection chamber may be provided with a device for blowing warm air in the aforementioned chamber so as to dry hands after the disinfection phase. In this case, the disinfection phase and drying phase are controlled automatically by a programmable control unit.

The disinfection chamber is preferably provided with a window enabling the user to see the position of his hands in the aforementioned chamber.

The bottom of the disinfection chamber can be inclined or funnel-shaped so as to facilitate the flow of condensate towards a low point, the latter being connected to a discharge conduit opening into a storage container.

The disinfection chamber advantageously comprises a raisable hood enabling, once raised, direct access to the interior of the aforementioned chamber.

Vents can be provided in the disinfection chamber so as to discharge any vapors that could ignite during the drying phase.

According to one implementation mode, the disinfection chamber is attached to a unit in which are positioned the mechanical components enabling its operation.

In another implementation mode, the apparatus comprises a unit in which the mechanical components are positioned enabling operation of the disinfection chamber, the aforementioned unit and the aforementioned chamber being joinable and detachable.

In an implementation variant, the disinfection chamber is provided with Xenon vapor lamps to disinfect their hands.

The apparatus object of the invention can be integrated into a table, a wall, etc.

### DESCRIPTION OF THE FIGURES

Other advantages and features of the invention will better appear upon reading the description of a preferred implementation mode that is going to follow, with reference to the accompanying drawings, made as indicative and non-limiting examples and in which:

FIG. 1a is schematic perspective view of a preferred implementation mode of the apparatus of the invention, the disinfection chamber being closed,

FIG. 1b shows the apparatus of FIG. 1 with the disinfection chamber open,

FIG. 2 is a schematic sectional view along A-A of the apparatus of FIG. 1a,

FIG. 3a is an enlarged view of the section B-B of the apparatus of FIG. 2,

FIG. 3b is a sectional view along C-C of the apparatus of FIG. 3a, only the disinfection chamber being shown,

FIG. 3c is a sectional view along D-D of the apparatus of FIG. 3b, only the disinfection chamber being shown,

FIG. 4 schematically shows a possible installation of the apparatus of the invention, the disinfection chamber being positioned on one side of a wall and the unit contain mechanical components on the other,

FIG. 5 schematically shows another possible installation of the apparatus of the invention, the disinfection chamber being positioned under the top of a table,

FIG. 6a is a sectional view of the disinfection chamber in implementation variant,

FIG. 6b is a sectional view along E-E of the disinfection chamber of FIG. 6a,

FIG. 7a is a sectional view of the disinfection chamber in another implementation variant,

FIG. 7b is a sectional view along F-F of the disinfection chamber of FIG. 7a,

FIG. 8a is a sectional view of the disinfection chamber in yet another alternative implementation variant,

FIG. 8b is a sectional view along G-G of the disinfection chamber of FIG. 8a.

### IMPLEMENTATION MODES OF THE INVENTION

The apparatus of the invention is designed to disinfect the hands of a user. It is particularly designed to be installed in a medical setting such as: hospital, medical office, operating room, etc. It can also be installed in a hotel, restaurant, factory, home, etc.

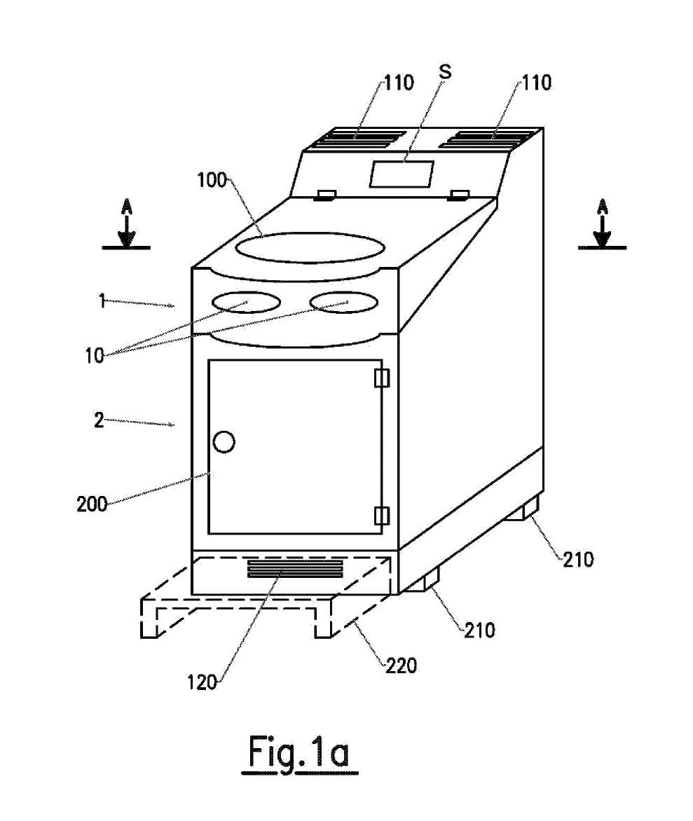

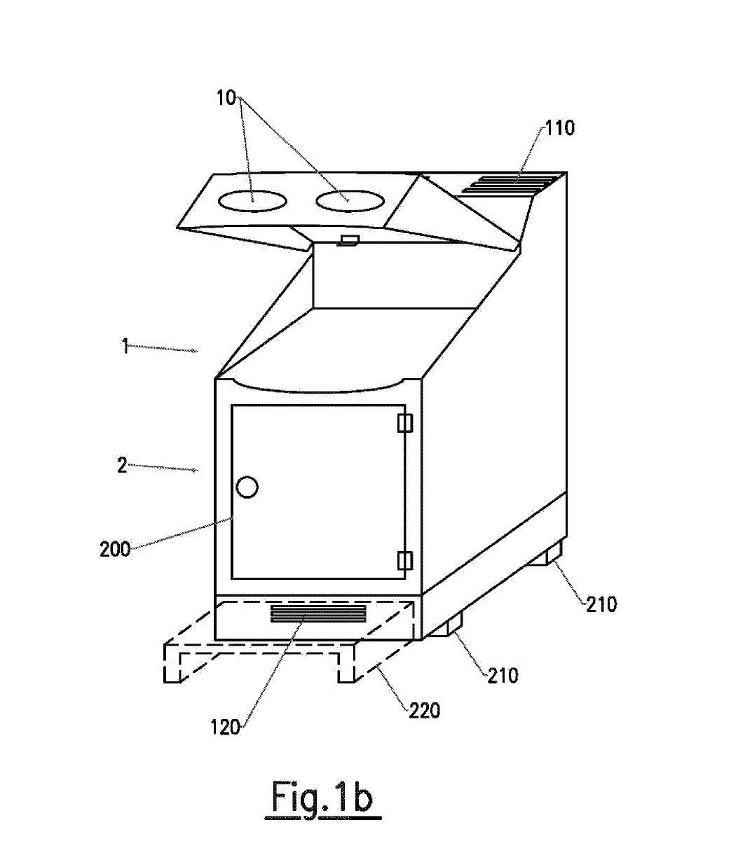

Referring to FIGS. 1a and 1b, the apparatus is composed of an upper part forming a disinfection chamber 1 and a lower part 2 forming a unit wherein the mechanical components are arranged (pump, blower, . . . ) for the operation of the aforementioned chamber. The latter advantageously comprises a tilting hood, articulated by means of hinges with a horizontal axis, and enabling, once raised (FIG. 1b), a direct access to the interior of the cavity. This is particularly useful during technical maintenance and/or during cleaning of the chamber. Similarly, the unit 2 is provided with a door 200 articulated by means of hinges with a vertical axis, and enabling direct access to the mechanical components required for the operation of the chamber 1. If these components generate mechanical vibrations, the unit 2 preferentially rests on anti-vibration supports 210, of the type made of rubber. And in the case where these mechanical components generate substantial noises, then the walls of the unit 2 can be covered with sound insulation such as rockwool, cork, polyester, “sandwich” panel, etc.

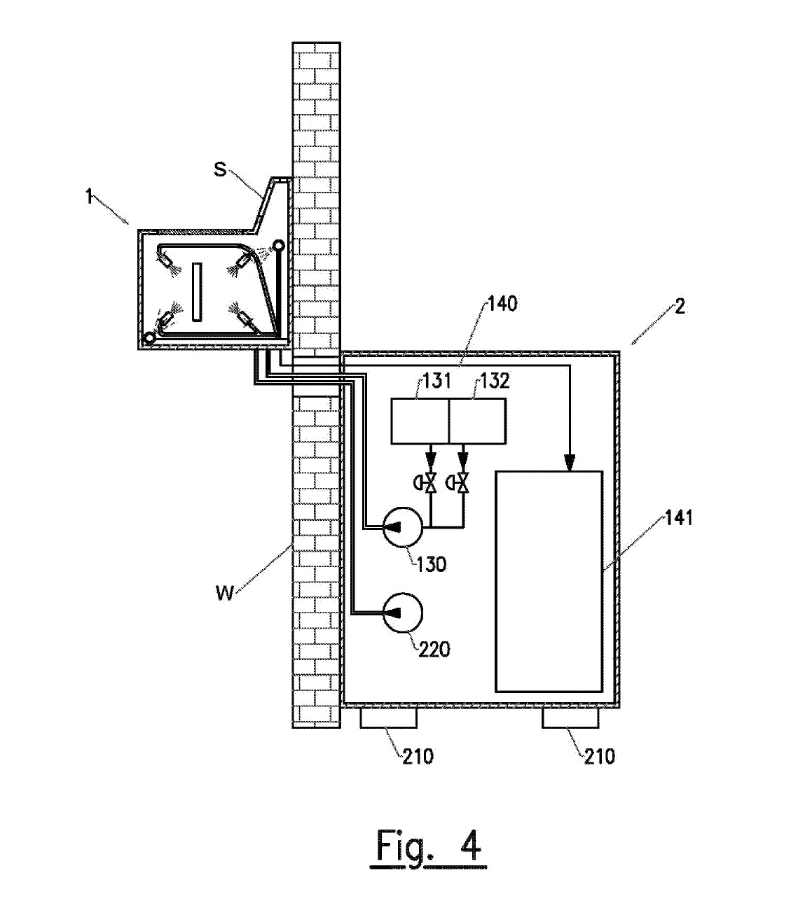

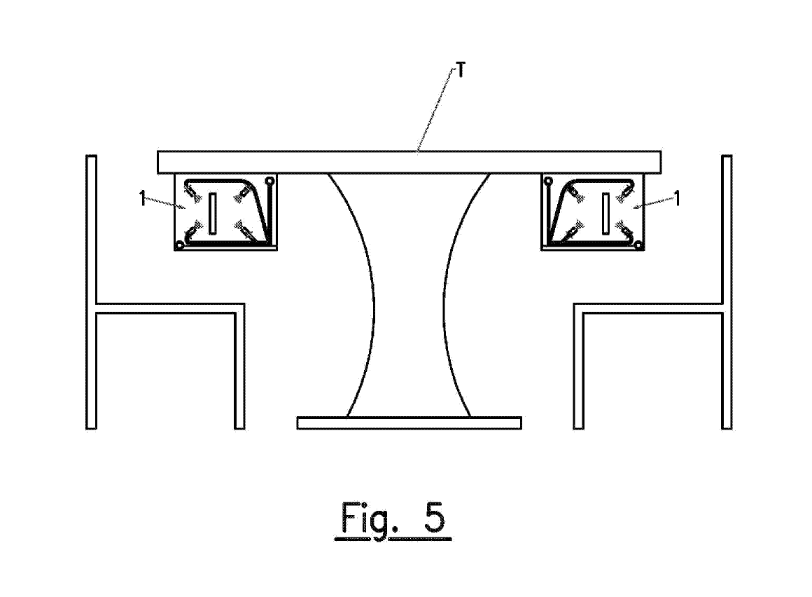

In the preferred implementation example shown in FIGS. 1a and 1b, the device is in the form of a terminal designed to be placed on the ground, the aforementioned terminal having a height of approximately 150 cm, a width approximately 60 cm and a depth of approximately 45 cm. This terminal is preferably formed of a metallic structure covered with metal. In this case, the chamber 1 is directly attached on the unit 2. Other shapes and dimensions can of course be provided. In particular, it is expected that the chamber 1 and unit 2 be joinable and detachable. Referring to the example of FIG. 4, it is thus possible to have a room on one side of a wall W (opening for example into a corridor, lobby, etc.) and the unit 2 of the other side of the aforementioned wall (opening for example into a technical room). This solution can be used in the case where the unit 2 contains mechanical components that are noisy and/or susceptible to generate vibrations. One can also provide for integrating the apparatus, and notably the chamber 1, directly into a wall, etc. Referring to FIG. 5, the chamber 1 is for example integrated into a table T, under the top of the aforementioned table. Thus when people are around the table T, they have the option of inserting their hands into the chamber 1 in order to disinfect them prior to and/or after their meal.

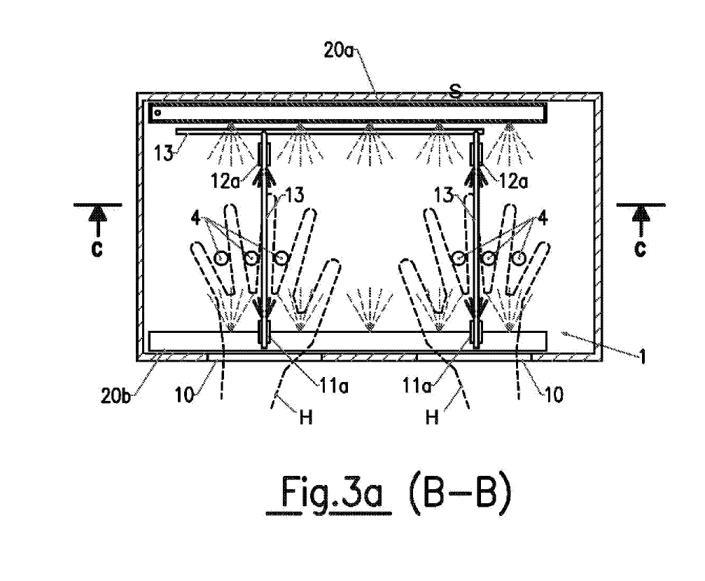

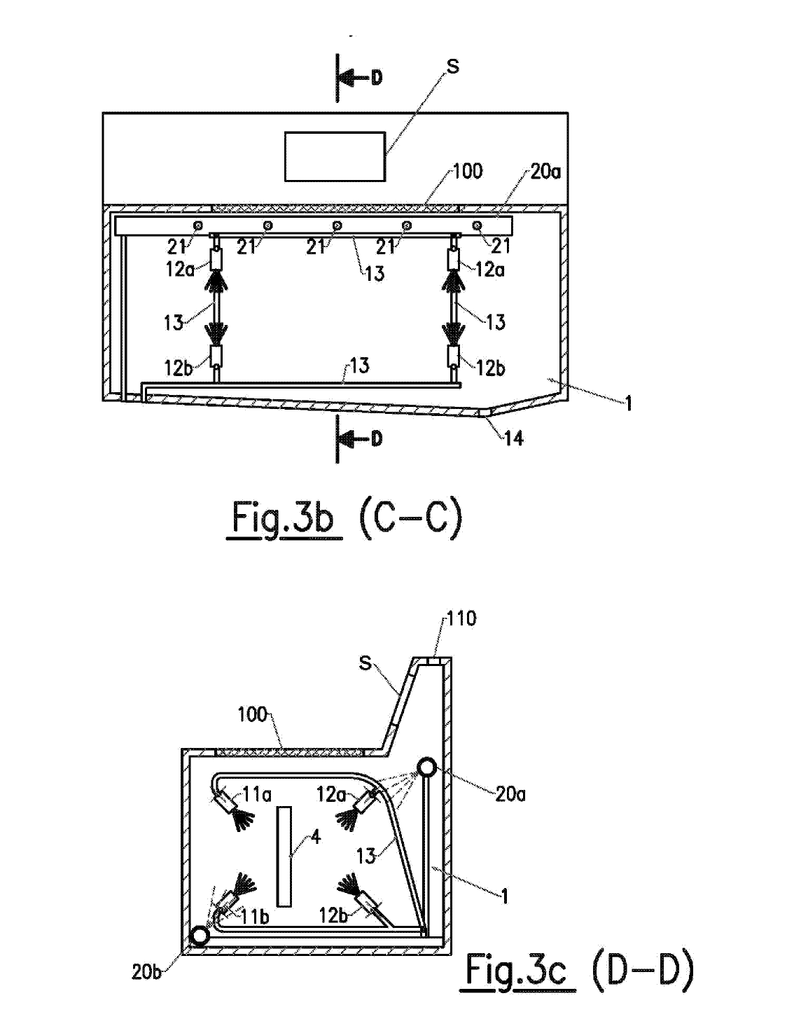

In practice, the disinfection chamber 1 comprises a partially sealed cavity in the center of which is positioned a device to spray a disinfectant product. The chamber 1 comprises at least an opening 10, preferably two, through which the user introduces his hands H (FIG. 3a). These openings 10 are positioned on the front of the apparatus. They are advantageously elliptical in order to minimize the dispersions of disinfectant product during its dissemination phase. The openings 10 may however be circular, rectangular, or any other shape enabling the user to introduce his hands. Similarly, one can provide a single opening large enough so that the user can introduce his two hands. Referring to FIGS. 1a and 1b, a step 22 (shown in phantom) can be positioned at the bottom of the front of the unit 2, to enable children or small people to properly introduce their hands into the chamber 1 through the openings 10.

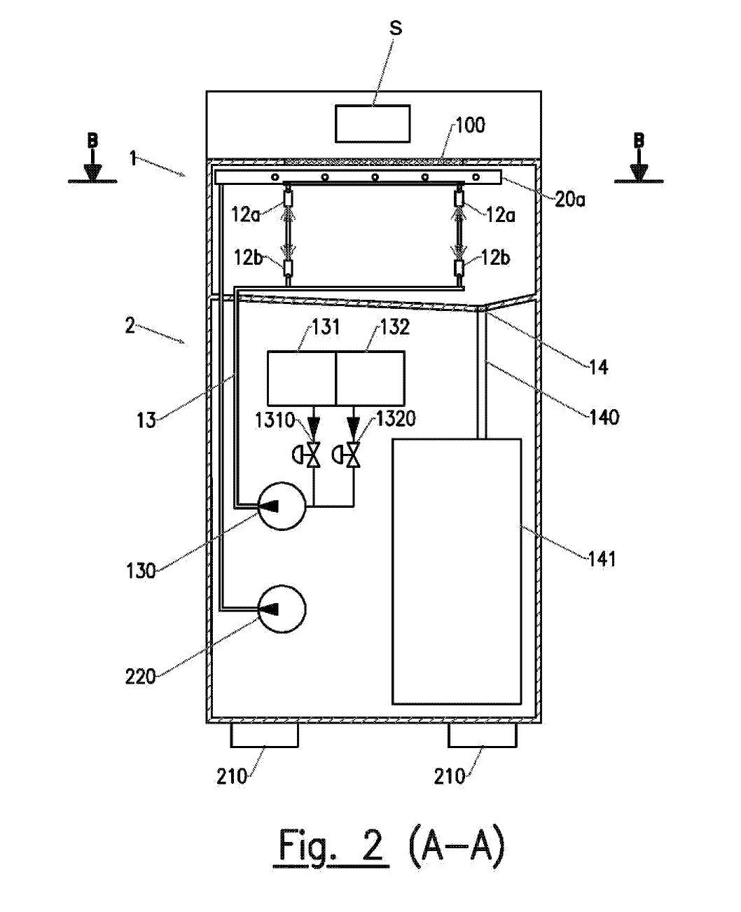

Referring to FIGS. 2 and 3b, the bottom of the chamber 1 is inclined or funnel-shaped so as to facilitate the flow of condensate of the sprayed disinfectant spray product, towards a low point 14. The latter is connected to a discharge conduit 140 leading into a storage container 141 that may be a sealed container recycling. The container 141 is positioned in the unit 2.

Referring specifically to FIGS. 2, 3a, 3b and 3c, the device for spraying the disinfectant product comprises nozzles 11a, 11b, 12a, 12b positioned so as to circulate the aforementioned product on the front and on the back of hands. Thus there are nozzles 11a, 12a placed in the upper part of the chamber 1, above the hands H and nozzles 11b, 12b placed in the lower part of the aforementioned chamber, below the aforementioned hands. In practice, each nozzle comprises a spray head producing a diverging spray. The design of these nozzles is well known to the person of skill in the art and will not be described here with more detail.

The disinfectant used is for example a hydroalcoholic liquid composition, a sodium hypochlorite or povidone iodine based liquid based composition, or with any other disinfectant suitable for the person of skill in the art.

According to an advantageous feature of the invention, the dispersion of the disinfectant product made not only from the fingertips to the wrist, but also from the wrist to the fingertips. In order to carry this out, nozzles 11a, 11b are positioned in front of the chamber 1 and oriented so as to distribute the disinfectant product to the rear portion of the aforementioned chamber. The dispersion is thus made from the wrist to the fingertips (FIGS. 3a and 3c). Other nozzles 12a, 12b are positioned in the rear part of the chamber 1 and oriented so as to disperse the disinfectant product to the front part of the aforementioned chamber. The dispersion is thus made from the fingertips to the wrist (FIGS. 3a and 3c). In this configuration, it is thus possible to effectively treat the entire surface of hands.

The nozzles 11a, 11b, 12a, 12b are attached on conduits 13 made of 316L stainless steel or medical plastic and shaped in the form of an arch in the interior of the chamber 1 (FIG. 3c). Referring to FIG. 2, the conduits 13 are connected to a pump 130 itself connected to a reservoir 131 containing the disinfectant product. A storage container 132 can also be provided. Valves 1310, 1320 enable selection of one of the reservoirs 131, 132. In practice, these reservoirs 131, 132 are removable containers or tanks from 5 L to 20 L positioned in unit 2.

The apparatus also incorporates a device for blowing air into the chamber 1. Referring to the implementation mode of FIGS. 2, 3a, 3b and 3c, this device comprises tubes 20a, 20b placed in a chamber, the aforementioned tubes being connected to an independent blower 22 positioned in the unit 2. Each tube 20a, 20b comprises perforations 21 enabling generation of a flow of air in the interior of the chamber 1. A first tube 20a is placed at the bottom of the chamber 1, in the upper part of the latter. The perforations 21 of this first tube are oriented so as to generate an air flow substantially horizontal but slightly inclined towards the tops of hands M. A second tube 20b is placed at the entrance of the chamber 1, in the lower part of the latter. The perforations of this second tube are oriented so as to generate an air flow substantially horizontal but slightly inclined towards the underside of hands M. The spraying of the disinfectant product and the air flow combine to homogeneously fog, or cloud, the aforementioned product in the interior of the chamber 1.

The nozzles 11a, 11b, 12a, 12b are preferably oriented in the chamber 1 so as to disperse the disinfectant product in a spatial sector defined by a vertical plane and an inclined plane of 1° to 90°, preferably of 45° to 90° relative to the axis of flow of blown air. Under these conditions, the disinfectant product is mixed well with air to form a homogeneous micro-fog, misted or sprayed, able to more easily reach all surfaces of hands H including inside the fingers. The characteristics of the micro-fog depend on the dimensions (mainly diameter) of the orifices of the nozzles 11a, 11b, 12a, 12b and the injection pressure. In practice, the nozzles 11a, 11b, 12a, 12b are mounted on hinges so that they can guide their dispersion.

According to the invention and as that is shown in FIG. 3a, separator 4 are placed in the chamber 1 so as to force the fingers to automatically separate from each other when the user inserts his hands H into the aforementioned chamber 1, and enable homogeneous distribution of the disinfectant product between the phalanges. The separators 4 are directly positioned in the disinfection chamber 1. The interior of the disinfection chamber 1 is devoid of a barrier between the separators 4 and the spaying device, so that the disinfectant product can be sprayed directly on the hands of the users. No obstacles, of the guide type described in the document U.S. Pat. No. 3,220,424 (NELSON), thus comes to surround the separators 1 and the hands. In practice, the separators 4 are vertical rods positioned in the chamber 1, at the openings 10, the spacing between the aforementioned rods defining the spacing of the fingers. These rods can be attached by welding, screwing or otherwise, or the horizontal bars placed transversely in the chamber 1 so as to link the two shorter sides of the aforementioned chamber. The rods have for example a diameter of between 3 mm and 10 mm and are spaced apart by a distance of between 2 cm and 4 cm. For each hand, three separators can suffice: a first designed for positioning between the little and ring fingers; a second designed for positioning between the ring and the major; a third designed for positioning between the major and index. One can foresee a fourth spacer designed for positioning between the forefinger and the thumb. But the thumb being already naturally separated from the index, this fourth spacer is not necessary. The separators 4 can also directly incorporated a device to spray disinfectant product directly between the fingers.

Referring to FIGS. 1a, 2, 3b and 3c, the chamber 1 is preferably provided with a window 100 enabling the user to see the position of his hands H in the aforementioned chamber in order to correctly position his fingers between the separators 4. The window 100 is arranged substantially at the center of the raisable hood of the chamber 1. It can be oval, round, rectangular, etc. In order to further facilitate the guiding of hands M, a lighting device is positioned in the interior of the chamber 1, the light being activated while the apparatus is turned on or while the user has his hands H positioned in the chamber 1. For this purpose, sensors can be provided to detect the presence of hands H in the chamber and activate the lighting.

When the user inserts his hands H into the chamber 1 and his fingers separated, then all the rough parts that lie between the fingers are always accessible and can be disinfected with the disinfectant product, which apparatus known in the prior art does not enable. Given that the area between the fingers is one where a maximum of bacteria accumulates, the apparatus object of the invention is more effective than those of the prior art.

The chamber 1 is preferably provided with a device for blowing warm air in the aforementioned chamber in order to dry the hands H after the disinfection phase. The apparatus thus combines the functions of disinfection and drying without the user having to rub his hands as is the case in most of the apparatus known in the prior art. Preferentially, the device for blowing warm air is the same as that used to blow the air during the phase of spraying the disinfectant product. Thus, the tubes 20a, 20b remain connected to the independent blower 200, but a heating resistance placed between the aforementioned tubes and the aforementioned blower is activated in order to heat the blown air. The tubes 20a, 20b can be mounted in rotation approximately their longitudinal axis and automatically placed in rotation in a back-and-forth movement by means of an electric motor, so that the airflow sweeps the entire chamber 1 and improves the drying of the hands. Any other equivalent device for blowing warm air can be used by the person of skill in the art.

The drying phase follows the disinfection phase, these two phases being controlled automatically by a programmable control unit. In practice, sensors are provided in order to detect the presence of hands H in the chamber 1, the control unit automatically triggering the cycle of disinfection-drying. The control unit can comprise an electronic card, a programmable login controller, one or multiple processors or microprocessors configured to execute one or multiple programs, subroutines, firmware or any other equivalent software in order to manage the operation of the unit. Referring to FIGS. 1a and 1b, a screen S is provided on the apparatus so as to indicate, to the user, the active phases and the treatment time remaining. This screen S can be equipped with a touch keyboard or otherwise and enables the user to choose a program from among multiple stored in the memory area of the control unit.

Referring to FIGS. 1a, 1b and 3c, it is advantageous to provide vents 100 in the chamber 1 so as to remove possible vapors that could ignite during the drying phase. Indeed, the spraying of the disinfectant product being capable of generating alcohol vapors, there is a risk that the latter will ignite when placed in contact with the warm drying air. The vents 110 are made in the top or on the sides of the chamber 1. In addition, these vents 110 enable discharge of the warm air during the drying phase. To improve the extraction of inflammable vapors and/or warm air, the vents 100 can be combined with a forced ventilation system.

Similarly, it is advantageous to provide vents 120 in the unit 2 so as to ensure a good air circulation in the aforementioned unit for an efficient cooling of the various pumps, motors and other electric heating resistance.

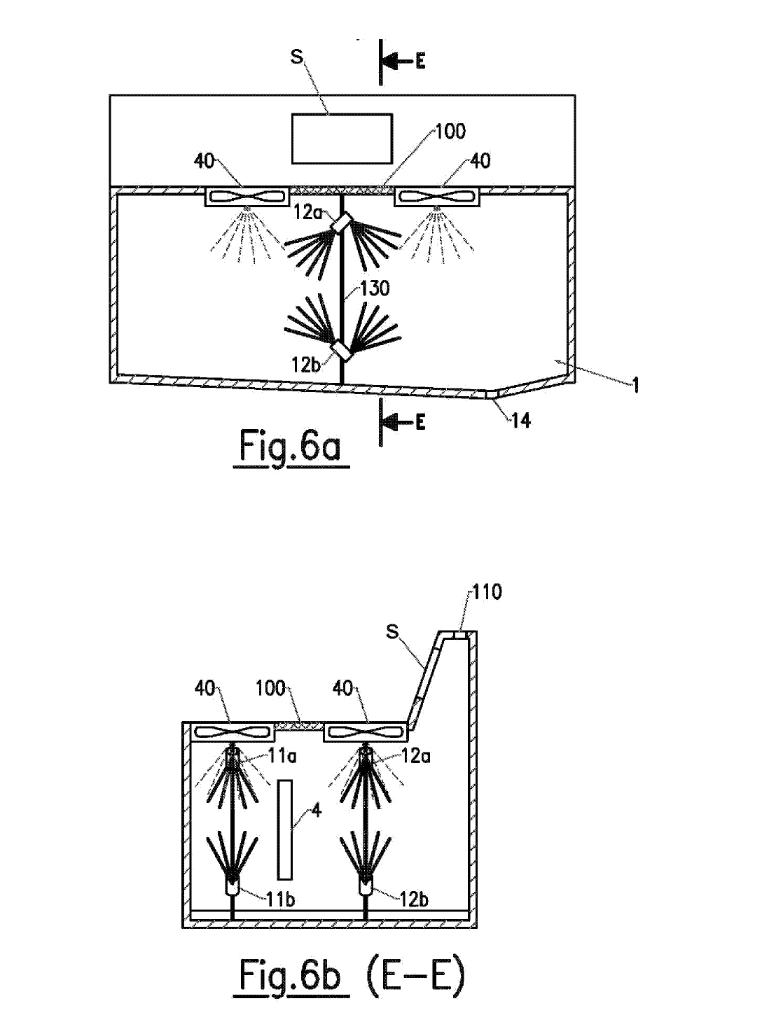

In the implementation mode shown in FIGS. 6a and 6b, the device for blowing air comprises fans 40 placed in the chamber 1. In practice, these fans 40 are incorporated in the hood of the apparatus and the airflow that they generate is substantially vertical, directed towards the base of the chamber 1. As described previously, the device for spraying the disinfectant product comprises nozzles 11a, 11b, 12a, 12b placed so as to disperse the aforementioned product on the front and the back of the hands. Preferentially, two columns of nozzles, respectively 11a-11b and 12a-12b are attached on the vertical rods 130. Each nozzle comprises a double pulverization head each producing a spray divergent towards each of the hands of the user. In accordance with the invention, the dispersion of the disinfectant product is carried out in a spatial sector defined by a vertical plane and an inclined plane of 1° to 90°, preferentially 45° to 90°, with respect to the axis of the flow of blown air. Nozzles 11a, 11b, 12a, 12b are connected to the pump 130 itself connected to the reservoirs 131 and 132.

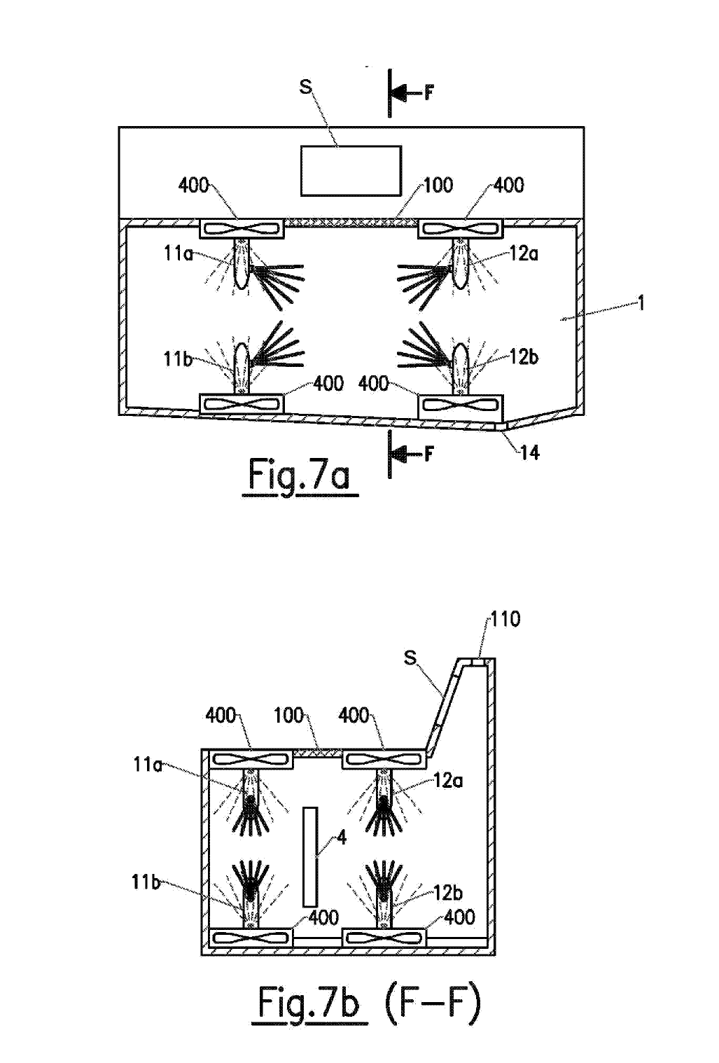

In the implementation mode shown in FIGS. 7a and 7b, the device for blowing air comprises fans 400 is placed in the chamber 1. In practice, these fans 400 are incorporated in the hood of the apparatus and in the bottom of the chamber 1. The air flow generated by these fans 400 is substantially vertical, directed downwards (for the fans above) or up (for the fans below). In this implementation mode, the nozzles 11a, 11b, 12a, 12b are mounted on supports directly attached on the axis of the fans 400. In accordance with the invention, the dispersion of the disinfectant product is carried out in a space segment defined by a vertical plane and an inclined plane of 1° to 90°, preferably of 45° to 90°, with respect to the axis of the flow of blown air.

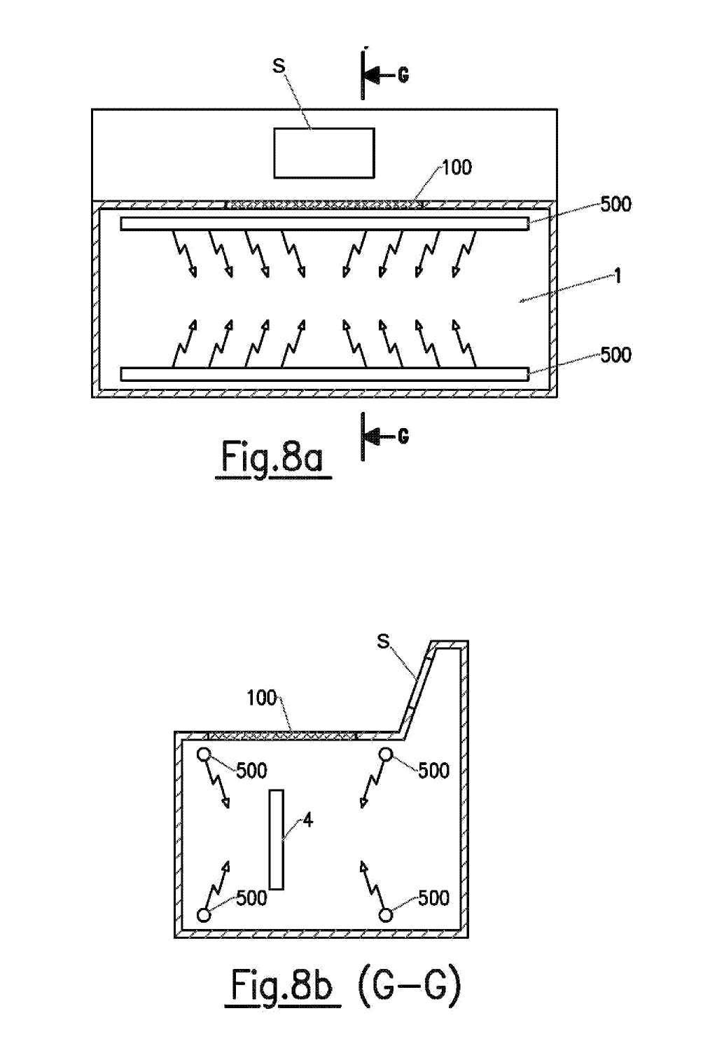

In the implementation mode shown in FIGS. 8a and 8b, the spaying device is replaced by Xenon vapor lamps 500, or any other type of lamp suitable for hand disinfection. These lamps 500 are positioned at the top and the bottom of the chamber 1 so as to treat the front and back of hands. The pulsed light from such lamps is an intense flash that causes the systematic breakdown of membranes and nuclei of all bacteria and viruses, preventing their reproduction. In this case, lamps 500 ensure that no contaminated surface can escape disinfection. The window 100 will optionally be treated to protect the user against possible adverse effects associated with the pulsed light.

1. An apparatus for disinfecting the hands of a user, the apparatus comprising:
a disinfection chamber,
an opening through which the user introduces his hands into the disinfection chamber,
separators placed in the disinfection chamber so as to force the fingers to separate from each other when the user inserts his hands into the chamber,
characterized by the fact that the disinfection chamber is provided with a device for spraying a disinfectant product in the chamber and a device for blowing air into the chamber, the two devices operating at the same time,
and by the fact that the device for spraying comprises nozzles enabling dispersion of a disinfectant product in the flow of blown air, the dispersion of the disinfectant product being carried out in a spatial sector defined by a vertical plane and an inclined plane of 1° to 90°, preferably of 45° to 90°, with respect to the axis of the air flow.

2. An apparatus according to claim 1, wherein the separators are vertical rods positioned in the disinfection chamber, the spacing between the rods defining the separation of the fingers.

3. An apparatus according to claim 1, wherein the separators incorporate a device for spraying the disinfectant product directly between the fingers.

4. An apparatus according to claim 1, comprising two elliptical openings through which the user inserts his hands into the disinfection chamber.

5. An apparatus according to claim 1, wherein the spaying device comprises nozzles dispersing a disinfectant product, the nozzles being arranged in the disinfection chamber so as to disperse the product on the front and back of the hands, the dispersion being carried out from the fingertips to the wrist and from wrist to the fingertips.

6. An apparatus according to claim 1, wherein the disinfection chamber is provided with a device for blowing warm air in the chamber so as to dry hands after the disinfection phase.

7. An apparatus according to claim 6, wherein the disinfection phase and drying phase are automatically controlled by a programmable control unit.

8. An apparatus according to claim 1, wherein the disinfection chamber is provided with a window enabling the user to see the position of his hands in the chamber.

9. An apparatus according to claim 1, wherein the bottom of the disinfection chamber is inclined or funnel-shaped so as to facilitate the flow of condensate to a low point, the latter being connected to a discharge conduit opening into a storage container.

10. An apparatus according to claim 1, wherein the disinfection chamber comprises a raisable hood enabling, once raised, direct access to the interior of the chamber.

11. An apparatus according to claim 1, wherein the disinfection chamber comprises vents.

12. An apparatus according to claim 1, wherein the disinfection chamber is attached to a unit in which are positioned the mechanical components enabling operation of it.

13. An apparatus according to claim 1, comprising a unit in which are arranged the mechanical components enabling operation of the disinfection chamber, the unit and the chamber being joinable and detachable.

14. An apparatus according to claim 1, wherein the interior of the disinfection chamber is free of obstacles between the separators and the spraying device, so that the disinfectant product can be directly sprayed on the hands of the user.

15. An apparatus according to claim 1, wherein the disinfection chamber is provided with Xenon vapor lamps in order to disinfect the hands.
